(19) United States
(12) Patent Application Publication
(10) Pub. No.: US 2006/0018942 A1
(43) Pub. Date: Jan. 26, 2006

(54) Polymeric microbeads having characteristics favorable for bone growth, and process including three dimensional printing upon such microbeads

(76) Inventors: Charles William Rowe, Medford, MA (US); Jie Cai, Newtown, PA (US); Andrea B. Caruso, Long Branch, NJ (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/139,971

(22) Filed: May 31, 2005

Related U.S. Application Data
(60) Provisional application No. 60/575,484, filed on May 28, 2004.

(51) Int. Cl. A61F 13/00

(52) U.S. Cl. 424/422

(57) ABSTRACT

The invention includes a method of three dimensional printing comprising manufacturing microbeads by an emulsion solvent extraction/evaporation process, followed by three dimensional printing onto powder layers comprising the microbeads. The invention also includes polymeric microbeads containing a bioactive substance or Active Pharmaceutical Ingredient, particularly a substance which stimulates the formation of bone, such as members of the statin family, or growth factors. The microbeads further may contain within themselves smaller particles such as particles of members of the calcium phosphate family, thereby being osteoconductive. The invention also includes aggregates of any of such microbeads together with any of various other types of suitably-sized particles, such as discrete particles of osteoconductive material or porogens. The invention also includes porous biostructures including macrochannels and having advantageous packing of osteoconductive particles.

### CROSS-REFERENCE TO RELATED PATENT APPLICATIONS

This patent application claims the benefit of U.S. provisional patent application No. 60/575,484, filed May 28, 2004, the disclosure of which is herein incorporated by reference in its entirety.

### BACKGROUND OF THE INVENTION

1. Field of the Invention

This invention pertains to three dimensional printing upon microbeads which have characteristics useful for promoting bone ingrowth.

2. Description of Related Art

In three-dimensional printing, which is described in U.S. Pat. No. 5,204,055, three-dimensional articles have been manufactured by selectively joining powder particles together by application of a binder fluid in a layer-by-layer manner. FIG. 1 illustrates a typical three dimensional printing (3DP) process. Frequently, powder particles used in three dimensional printing have been prepared by milling, in which particles have been ground or rubbed between hard surfaces and each other, causing the particles to fracture and thereby form smaller particles. The particles produced by such a process have typically included a variety of sizes and a variety of shapes, which have frequently been irregular shapes due to the nature of fracturing. However, for three dimensional printing, for example, for the step of spreading or depositing the powder layer, it may be preferable that the particles have a somewhat regular (somewhat smooth or somewhat close to equiaxial) shape. In regard to polymeric particles in particular, milling of polymers has sometimes been performed at cryogenic temperatures to increase the brittleness of the polymer being milled, but the process has been laborious and time consuming and has provided only a low yield of desirably sized particles. Another method which has been used to prepare powders for use in 3DP has been spray-drying. Spray-dried powder particles have tended to be substantially spherical, but only some materials have been suitable to be formed into powder particles or microbeads by spray-drying.

In the pharmaceutical arts, microbeads for drug delivery have been created by an emulsion solvent extraction/evaporation process, which is illustrated in FIG. 2. In this process, an emulsion has been created comprising droplets of a discontinuous liquid phase surrounded by a continuous liquid phase. The droplets have been a first liquid which has been a first solvent such as an organic solvent containing the intended microbead material(s) such as polymer dissolved in it. The continuous phase has been a second liquid which has typically been water containing a surfactant. The emulsion of the two liquids has been maintained for a sufficient period of time so that the first solvent has gradually passed out from the droplets into the continuous phase, resulting in the formation of solid particles of the microbead material. This process or similar processes are described in U.S. Pat. No. 4,389,330; “Neutrophil activation by plasma opsonized polymeric microspheres: inhibitory effect of Pluronic 127,” by M. K. Springate et al., Biomaterials 21(2000) 1483-1491; “Morphology, drug distribution and in vitro release profiles of biodegradable polymeric microspheres containing protein fabricated by the double-emulsion solvent extraction/evaporation method,” by Y. Y Yang et al., Biomaterials 22(2001), 231-241; “Morphology and biodegradation of microspheres of polyester-polyether block copolymer based on polycaprolactone/polylactide/poly(ethylene oxide),” by D. Chen et al., Polymer International 49:269-276(2000); and “Tissue engineered microsphere-based matrices for bone repair: Designs and evaluation,” by Borden M D, Attawia M A, Khan Y, Laurencin C T., Biomaterials 2002; 23(2):551-559. A more precise version of this method has involved creating the droplets in the emulsion by dispensing individual droplets from an ink-jet printhead, as described in “Uniform Paclitaxel-Loaded Biodegradable Microspheres Manufactured by Ink-Jet Technology,” by Radulescu et al., Proc. Recent Adv. in Drug Delivery Sys., March 2003. The microbeads created by any such emulsion solvent extraction/evaporation process have generally been intended as a delivery vehicle for Active Pharmaceutical Ingredients, have typically been released or injected into the body as individual unconnected microbeads, and have so far not been available for or optimized for use as a powder in three dimensional printing.

In general, it is useful for bone repair implants to include members of the calcium phosphate family as a raw material for the formation of natural bone. Based on this principle, microbeads have been made which included within themselves either single or multiple smaller particles of calcium phosphate material. Such microbeads have been attached to other such microbeads such as by sintering at a temperature which just softens the polymer. Such microbeads are described in: “Tissue engineered microsphere-based matrices for bone repair: Designs and evaluation,” by Borden M D, Attawia M A, Khan Y, Laurencin C T., Biomaterials 2002; 23(2):551-559; “Bioactive, degradable composite microspheres: Effect of filler material on surface reactivity,” by Qui Q Q, Ducheyne P, Ayyaswamy P S, Ann N Y Acad Sci 2002;974:556-564; and “The merit of sintered PDLLA/TCP composites in management of bone fracture internal fixation,” by Lin F H, Chen T M, Lin C P, Lee C J, Artif. Organs 1999; 23(2):186-194, and U.S. Pat. No. 6,358,532. Notwithstanding, there remains a need in the art for microbeads which are both osteoconductive and also are able to stimulate the formation of bone. Furthermore, biostructures comprising such microbeads and providing detailed internal geometry such as macrochannels are additionally desirable, since macrochannels are known to be useful for fostering the ingrowth of bone. Also, it is always of interest to provide as much osteoconductive material as possible in such a biostructure, relative to the amount of polymer.

Accordingly, it is desirable to provide, for use in the three dimensional printing process, a supply of polymeric particles which is of well-controlled shape and size and with a high yield fraction of desirably shaped and sized particles. It is desirable to provide polymeric microbeads which contain a bioactive substance such as Active Pharmaceutical Ingredient, in particular an API or bioactive substance which stimulates the formation of bone. It is further desirable to provide polymeric microbeads which contain within the microbeads solid particles such as osteoconductive particles. It is desirable to provide a powder mixture or aggregate which comprises both the described microbeads and other types of particles. It is desirable for a biostructure to have macrochannels for the ingrowth of bone. It is desirable to provide a biostructure which, while being porous, contains a high fraction of osteoconductive material, relative to the amount of polymer.

### BRIEF SUMMARY OF THE INVENTION

The invention includes a method of three dimensional printing which comprises manufacturing microbeads by an emulsion solvent extraction/evaporation process and later performing three dimensional printing onto powder layers which comprise the microbeads. The droplets in the emulsion solvent extraction/evaporation process may have a size distribution which is determined by agitation parameters, liquid properties, etc., or the droplets may be formed having a controlled size and may be introduced into the other liquid.

The invention also includes polymeric microbeads which may contain within themselves a bioactive substance or Active Pharmaceutical Ingredient, particularly a substance which stimulates the formation of bone, such as members of the statin family, or growth factors. The microbeads further may contain within themselves smaller particles such as particles of members of the calcium phosphate family, thereby being osteoconductive. The microbeads may have internal porosity. The invention also includes aggregates of any of such microbeads together with any of various other types of particles, such as discrete particles of osteoconductive material, porogens, etc., all of which may be suitably sized.

The invention also includes biostructures made by or using the above aspects of the invention, which biostructures may be porous and may include macrochannels and may have advantageous packing of osteoconductive particles.

### BRIEF DESCRIPTION OF THE SEVERAL VIEWS OF THE DRAWING(S)

The invention is further described in the following Figures, in which:

FIG. 1 illustrates a conventional three dimensional printing process.

FIG. 2 illustrates the conventional emulsion solvent extraction/evaporation process.

FIG. 3 illustrates a microbead of the present invention.

FIGS. 4a and 4b illustrate biostructures of the present invention containing both large and small osteoconductive particles.

FIG. 5a illustrates the method of the present invention in flowchart form, and FIG. 5b illustrates some of the steps graphically.

FIG. 6 illustrates an experimentally determined particle size distribution for a batch of PCL particles made as described in Example 1.

FIG. 7 is a Scanning Electron Microscope micrograph of polycaprolactone (PCL) particles made as described in Example 1.

FIG. 8 illustrates an experimentally determined particle size distribution for a batch of PCL particles made as described in Example 2.

FIG. 9 illustrates an experimentally determined particle size distribution for a batch of PCL particles made as described in Example 3.

FIG. 10 illustrates an experimentally determined particle size distribution for a batch of PLGA particles made as described in Example 4.

FIG. 11 illustrates a simple shape made using three dimensional printing onto a powder of polymer microbeads made by the emulsion solvent extraction/evaporation process.

FIGS. 12a and 12b illustrate spray-dried lightly-sintered TCP particles before processing and after processing (which resulted in fracturing of the particles).

FIGS. 13a and 13b illustrate other, more robust TCP particles before processing and after processing.

FIG. 14 illustrates microbeads formed by air drying of droplets.

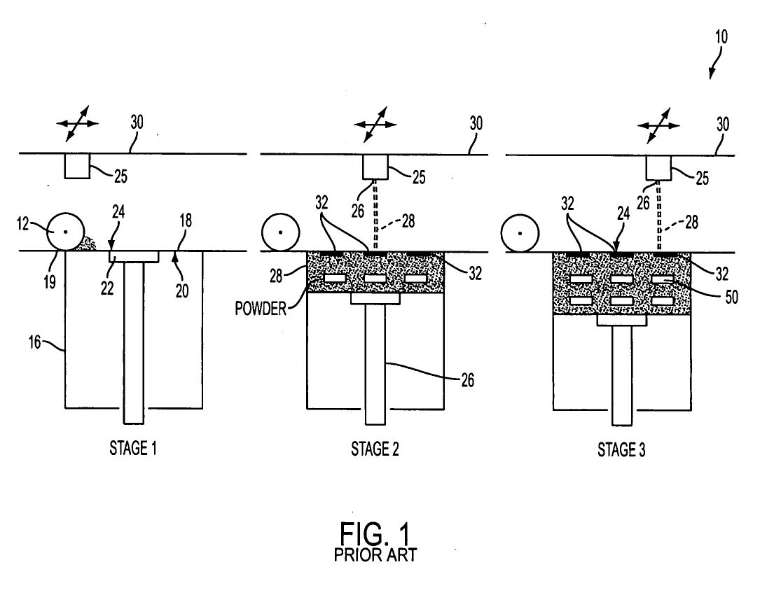

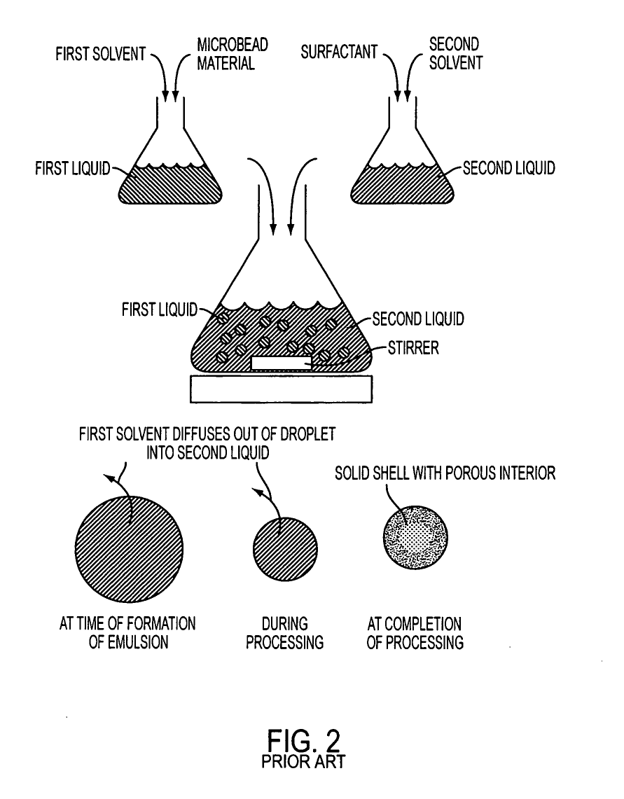

### DETAILED DESCRIPTION OF PREFERRED EMBODIMENTS

An aspect of the invention is microbeads themselves. The microbeads may be of a size suitable to be used as a powder in three dimensional printing, and may be of a size suitable to produce a biostructure having desirably sized pores. For example, the microbeads may have a size distribution such that the average size or most common size of the microbeads is somewhere in the range of from approximately 10 micrometers to approximately 300 micrometers. The width of the size distribution of the microbeads may be narrower than that range. The microbeads may be roughly spherical or equiaxial in shape. The surfaces of the microbeads may be smooth or may be locally wrinkled. Some embodiments of the invention are microbeads which have a substantially continuous, smooth outer surface, while other embodiments of the invention are microbeads which are porous, including having pores that break through the surface of the microbead.

Some embodiments of the invention are microbeads comprising just a single substance which is a polymer. Other embodiments are microbeads which comprise a polymer and also, within the microbead, particles of a substance which is osteoconductive. In any embodiment which contains particles of an osteoconductive substance, the particles of the osteoconductive substance may have a dimension larger than a dimension which causes a macrophage response in the body, such as greater than about 5 micrometers or greater than about 10 micrometers. At the same time, the particles of osteoconductive substance may be small enough so that a desired number of such particles can fit within a microbead. For example, if particles of an osteoconductive substance are present within a microbead, the number of such particles may range from one to several hundred.

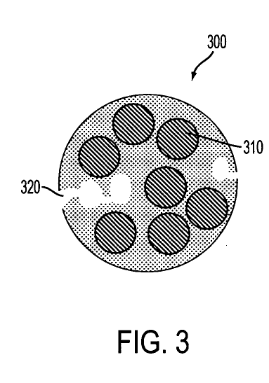

Such a microbead is shown in FIG. 3. FIG. 3 illustrates microbead 300, shown as being spherical, which may contain particles 310 of osteoconductive material and may also contain pores 320.

The osteoconductive material may be any member of the calcium phosphate family. In particular, beta tricalcium phosphate is believed to have desirable resorption characteristics. In other embodiments of the invention, calcium compounds such as other calcium phosphates, or other calcium compounds such as calcium sulfates and calcium carbonates are provided as the osteoconductive material. It is also possible that the osteoconductive material may be or may include still other ceramics or bioactive glass.

Some embodiments of the invention are microbeads which comprise a polymer and a bioactive substance such as an Active Pharmaceutical Ingredient (API) or growth factor. If an API, growth factor or other bioactive substance is contained within the microbead, such substance either may be distributed throughout (co-located with) the polymeric material of the microbead or may be in the form of identifiable particles within the microbead. In one embodiment of the invention, API is provided in the form of discrete particles within microbeads, wherein the API particles are not restricted by size.

In embodiments of the invention, any resorbable polymer can be blended or co-located with a bioactive substance such as an API. In this embodiment, the polymer can have a resorption characteristic in the bodily environment which provides a desired release characteristic of the bioactive substance.

A specific category of bioactive substance of interest is substances which stimulate the formation of bone, such as by stimulating the production of bone morphogenetic proteins. A known category of such substances is HMG-CoA reductase inhibitors, which includes members of the statin family. Statins, which were originally developed for the control of cholesterol, have also been found to be useful for stimulating the production of bone morphogenetic proteins. The presence of such substances in an implant gives the implant properties which are quite similar to the property of osteoinductivity. Possible members of the statin family which could be used in the present invention include, but are not limited to, lovastatin, simvastatin, pravastatin, fluvastatin, atorvastatin, cerivastatin, mevastatin, and combinations thereof. The statins may be used in the form of pharmaceutically acceptable salts, esters and lactones thereof. Lovastatin, as used in the invention, may be in either the beta hydroxylic acid form or the lactone form, or a combination of both forms. It is also possible for the microbead to contain growth factors as separate substances, or in conjunction with members of the statin family.

Of course, it is possible that the microbead comprise, or alternatively consist of, a combination of a polymer, a bioactive substance and particles of a material which may be osteoconductive.

As a result of the presence in the microbeads of any of various substances (certain API, growth factors, other suitable bioactive substances), the microbeads induce certain biological functions depending on the substance included in the microbeads. Such biological functions may result in the growth of bone. This property may be in addition to the attribute of osteoconductivity based on the microbeads containing particles of substances such as members of the calcium phosphate family.

The microbeads may comprise an Active Pharmaceutical Ingredient or other bioactive substance which is at least somewhat water-soluble. Such substance may be contained within the interior of the microbead, such as within discrete regions within the microbead. In such instance, it is even possible that water containing the water-soluble substance may be present in the microbead.

The microbeads may further comprise still other bioactive substances such as other categories of bioactives or Active Pharmaceutical Ingredients. Non-limiting categories of such substances include angiogenic agents, antibiotics, anesthetics and chemotherapeutic agents.

As far as polymeric materials, the microbeads may comprise almost any polymer which is soluble in at least one organic solvent. The polymer may be either resorbable or nonresorbable. In particular, the polymer may be any of a number of polyhydroxy acid, such as poly-lactic acid (PLA); poly lactic acid glycolic acid co-polymer (PLGA); poly caprolactone (PCL); poly(hydroxybutyric acid) and poly(hydroxyvaleric acid). Still other examples of polymers include poly(trimethylene carbonate); polyanhydrides; polyorthoesters; polyphosphoesters; polymers of acrylic acid or copolymers or derivatives thereof including esters, such as poly(methyl methacrylate); polyamides; polyvinyl ethers; polyvinyl esters; polyfumarate; polyvinylpyrrolidone; polyalkylene glycols; and polyalkylene oxides such as poly(ethylene oxide). The microbead material may include copolymers or terpolymers of any of the listed substances. The following polymers are suitable for making the biostructure: Polylactides; Polyglycolides; Epsilon-caprolactone; Polyhydroxyvaleric acid; Polyhydroxybutyric acid; other Polyhydroxy acids; Polytrimethylene carbonate; polyamines; vinyl polymers; Polyacrylic acid and their derivatives including esters; Polyethylene glycols; Polydioxanones; Polycarbonates; Polyacetals; Polyorthoesters; Polyamino acids; Polyphosphoesters; Polyesteramides; Polyfumerates; Polyanhydrides; Polycyanoacrylates; Poloxamers; Polyurethanes; Polyphosphazenes; Aliphatic polyesters; Poly(amino acids); Copoly(ether-esters); Polyalkylene oxalates; Polyamides; Poly(iminocarbonates); Polyoxaesters; Polyamidoesters; Polyoxaesters containing amine groups; Polyacetals; Polyalkanoates; Gelatin; Collagen; Elastin; Polysaccharides; Alginate; Chitin; Hyaluronic acid; Poly(L-lactic acid) (PLLA); Poly (DL-lactic acid); Poly-DL-lactide-co-glycolide (PDLGA); Poly(L-lactide-co-glycolide) (PLLGA); PLLA-co-GA; PLLA-co-GA 82:18; Poly-DL-lactic acid (PDLLA); PLLA-co-DLLA; PLLA-co-DLLA 50:50; PGA-co-TMC (Maxon B); Poly-p-dioxanone (PDS); PDLLA-co-GA (85:15); aliphatic polyester elastomeric copolymer; epsilon-caprolactone and glycolide in a mole ratio of from about 35:65 to about 65:35; epsilon-caprolactone and glycolide in a mole ratio of from about 45:55 to about 35:65; epsilon-caprolactone and lactide selected from the group consisting of L-lactide, D-lactide and lactic acid copolymers in a mole ratio of epsilon-caprolactone to lactide of from about 35:65 to about 65:35; Poly(L-lactide and caprolactone in a ratio of about 70:30); poly (DL-lactide and caprolactone in a ratio of about 85:15); poly(DL-lactide and caprolactone and glycolic acid in a ratio of about 80:10:10); poly(DL-lacticde and caprolactone in a ratio of about 75:25); poly(L-lactide and glycolic acid in a ratio of about 85:15); poly(L-lactide and trimethylene carbonate in a ratio of about 70:30); poly(L-lactide and glycolic acid in a ratio of about 75:25);. The polymer can also be copolymer or terpolymer. It can be a blend of two or more individual substances mixed together. The microbead material also may include or may be a comb polymer as described elsewhere herein.

Any of the polymers provided supra may be present in the microbeads in any concentration and in any combination. Any of the components provided supra may be present in the microbeads in any amount and in any combination.

Another aspect of the invention is an aggregate or powder mixture which comprises the described microbeads. In addition to the described microbeads, the aggregate or powder mixture may comprise any one or more, in any combination, of the following: other microbeads having other characteristics; discrete particles of osteoconductive material (which may be or include beta tricalcium phosphate, other calcium phosphates, calcium sulfates, calcium carbonates, other calcium compounds, other ceramics, bioactive glass, etc.); discrete particles of one or more porogen substance(s); and discrete particles of Active Pharmaceutical Ingredient. It is possible that any or all of the constituents of the aggregate may be size-classified so as to provide particles and/or microbeads having a size or size distribution which is appropriate for the powder-spreading or other operations which take place during three dimensional printing. Such particle/microbead size and size distribution may be different from the size and size distribution which are appropriate for other powder-based manufacturing methods. (For example, molding may likely be able to tolerate a less-closely-controlled size and size distribution than three dimensional printing.) For example, the particle size distribution of the various constituents of the powder mixture may all be controlled such that substantially all of the particles or microbeads are within a size range of 20 to 100 micrometers. More generally, the particle size distribution of the various constituents of the powder mixture may all be controlled such that substantially all of the microbeads and particles of the aggregate are between a maximum size and a minimum size, and the ratio of the maximum size to the minimum size is less than approximately 5. The average size of the particles and microbeads in the aggregate (along with the characteristics of porogen particles if used) may be selected so as to produce a desired average pore size in the final biostructure. The aggregate may contain more than one kind of microbead. For example, microbeads containing an API or bioactive substance which is soluble in an organic solvent may be one kind of microbead. Microbeads containing an API or bioactive substance which is at least somewhat water-soluble may be physically different in that they may contain internal regions with the API or water. The polymer may be different among the two types of microbeads. Both types of microbeads may be present in the aggregate.

Another aspect of the invention is a biostructure made using the described microbeads or the described aggregate or powder mixture. The use of certain forms of the aggregate can result in a particular situation as far as the sizes of osteoconductive particles in the final biostructure. As described in more detail elsewhere herein, the biostructure may be made from a powder mixture which comprises discrete particles of an osteoconductive substance in a particle size range appropriate for 3DP, and which also comprises the described microbeads, which may be of a similar size but may contain within themselves smaller osteoconductive particles. Thus, it is possible that the biostructure may contain particles of an osteoconductive substance which are in a first size range appropriate to be individual particles of the powder mixture, and further may contain particles of the same or different osteoconductive substance which are in a second size range, generally smaller than the first size range. The smaller osteoconductive particles may be of a size such that a plurality of the particles of the second size range would be able to be contained inside a microbead whose size is approximately the first size range. Thus, the size distribution of the overall population of osteoconductive particles in the biostructure may be bimodal. In such a bimodal distribution, the mode at the larger of the two sizes may be typical of individual particles of osteoconductive substance which are in the powder mixture, and the mode at the smaller of the two sizes may be typical of particles of osteoconductive substance which are contained within microbeads. The sizes of both types of osteoconductive particles may be sufficiently large so as to avoid causing a macrophage response in the body of the recipient. A typical requirement for such purpose is that the particles be larger than approximately 10 micrometers.

Depending on the nature of the manufacturing processes described elsewhere herein, material in the microbeads may undergo various degrees of rearrangement by the time the finished biostructure has been produced. Thus, in the finished biostructure, osteoconductive particles which were of a size appropriate to fit inside microbeads and osteoconductive particles which were comparable to the size of microbeads may be in close or not-so-close intermingling with each other.

In one embodiment, the biostructure may have a microstructure which includes recognizable microbeads joined to other microbeads or to other particles (such as osteoconductive particles or any type of particle) by films, necks or other joining structures which comprise polymeric material. Some of the osteoconductive particles (the larger size range of osteoconductive particles) may not be contained inside any other recognizable shape, while at least some of the other osteoconductive particles (the smaller size range of osteoconductive particles) may be contained inside overall shapes which are substantially the shape of a microbead. It is possible that there can be some identifiable microbeads while other polymeric material is not recognizable as being in the shape of a microbead. The material forming the necks or joining structures may be substantially the same polymeric or polymer-plus-small-osteoconductive-particle material which makes up the microbeads.

In another embodiment it is possible that the biostructure may have the geometry of a plurality of thin films which are irregularly shaped and perforated and which hold particles of the osteoconductive material of any size described herein. In this situation, the various sizes of particles of osteoconductive material may be more thoroughly intermingled with each other.

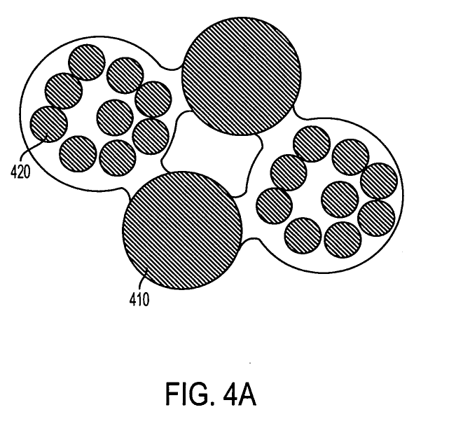

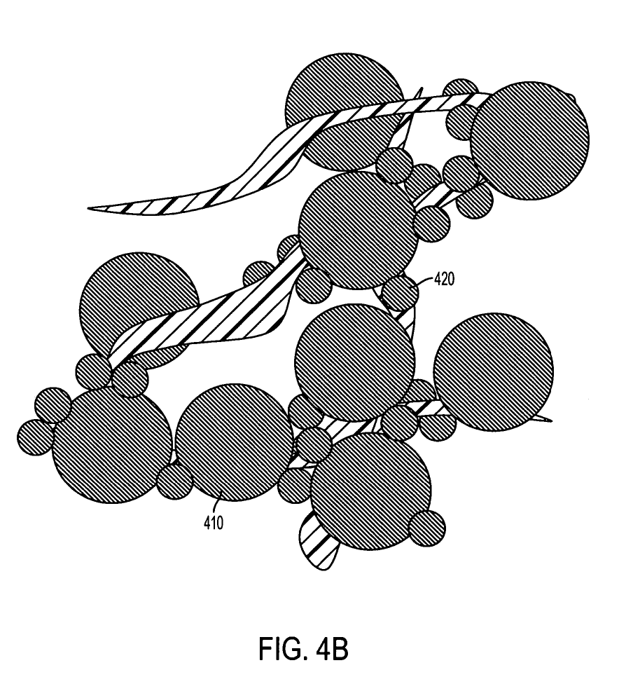

Various such biostructures are illustrated in FIGS. 4a and 4b, including large osteoconductive particles 410 and small osteoconductive particles 420.

In any microstructure in which osteoconductive particles of more than one size range are intermingled with each other to at least some degree, the existence of a bimodal particle distribution is helpful for achieving an efficient packing of any material of interest into a given overall geometry. In such a packing, the large particles can arrange themselves in generally any arrangement which provides reasonably efficient packing, and then the small particles can essentially fill in much of the empty space between the large particles. For example, the most common small particle size may be somewhere between approximately one-tenth and one-third of the most common large particle size, such as for example approximately one-quarter of the most common large particle size. In this case, in which the overall biostructure is itself porous, packing refers to the makeup of the solid material of the biostructure, not to the pores of the biostructure themselves. This can achieve a greater packing of osteoconductive particles in the solid regions of the biostructure than would be achieved with a more uniform distribution of particle sizes of osteoconductive material. This can provide more osteoconductive material to the body of the patient and correspondingly less polymeric material (which at some point has to break down into decomposition products which then need to be removed or cleared by the body of the patient).

The biostructure can contain microbeads which are at least somewhat recognizable as microbeads whose polymer material is one composition, and can further contain either somewhat recognizable microbeads or material from microbeads whose polymeric material is another composition. For example, the microbeads which contain water-soluble API or bioactive substance may be more recognizable as intact microbeads, and the polymer in those microbeads may be relatively more resistant to softening as a result of exposure to at least some organic solvent, as compared to the polymer in the microbeads which do not contain such API or bioactive substance. As a result, the microbeads which are more resistant to softening can be contained in the final biostructure in a relatively intact manner which means that material in those microbeads can be protected from exposure to water which might occur during the later stages of the manufacturing process, and therefore such water-soluble substances can still be contained in the biostructure. For example, in general, polymers which soften and flow relatively easily are those with a non-crystalline structure and with a low molecular weight. Polymers which are relatively more resistant to softening and flowing are those with a more crystalline structure and with a higher molecular weight. A polymer which is less resistant to dissolving in organic solvents and to softening is PLGA-DL. Examples of polymers which are more resistant to softening are polycaprolactone (PCL) and PLGA-L.

In a biostructure, the macrostructure can refer to geometric structure which is on a size scale larger than several of the largest microbeads or particles in the aggregate; or, if such particles are extremely small, macrostructure can refer to geometric structure on a size scale larger than about 50 micrometers. In terms of macrostructure, the biostructure may have channels or internal features of almost any degree of geometric complexity consistent with the three dimensional printing process (i.e., the ability to remove unbound powder after completion of printing). Macrochannels, internal voids or other macroscopic features may be connected to the exterior of the biostructure and may have cross-sectional dimensions of an appropriate size scale (a sufficiently large number of powder particle diameters such that the unbound powder particles can be removed from the channel, void or feature). The biostructure may have an overall exterior shape that includes geometric complexity such as undercuts, recesses, interior voids, and the like, provided that the undercuts, recesses, interior voids, and the like have access to the space outside the biostructure. The biostructure may be shaped appropriately so as to replace a particular bone or bones or segments of bones or spaces between bones or voids within bones, which may be unique for a particular patient. The biostructure may have holes or passageways or channels that may each have a cross-section that is substantially constant. Alternatively, the holes passageways channels or other macrostructural features may be curved, have changes of direction, have varying cross-section, and the like, and can branch to form other passageways or channels or holes or can intersect other passageways or channels or holes. It is possible to have intersections of multiple channels, even including intersections of three co-planar or non-coplanar channels at a common intersection point. It is also possible to have dead-end channels which do not intersect any other channel or feature. It is possible to have grooves or dimples which exist on exterior surfaces of the biostructure. All such features are believed to be helpful for the ingrowth of bone. Cross-sectional dimensions of any such feature may range from about 50 to about 2000 microns and more typically range from about 200 to about 700 microns in size.

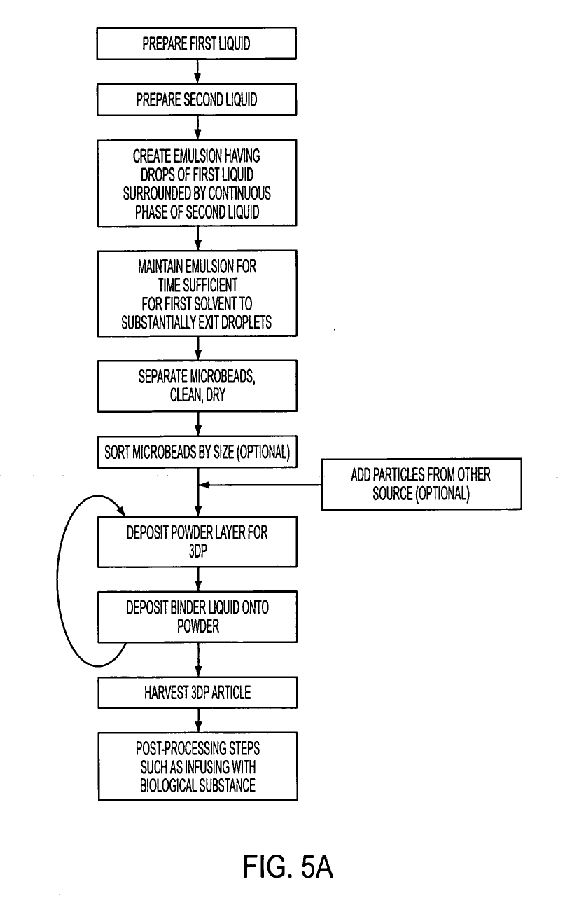

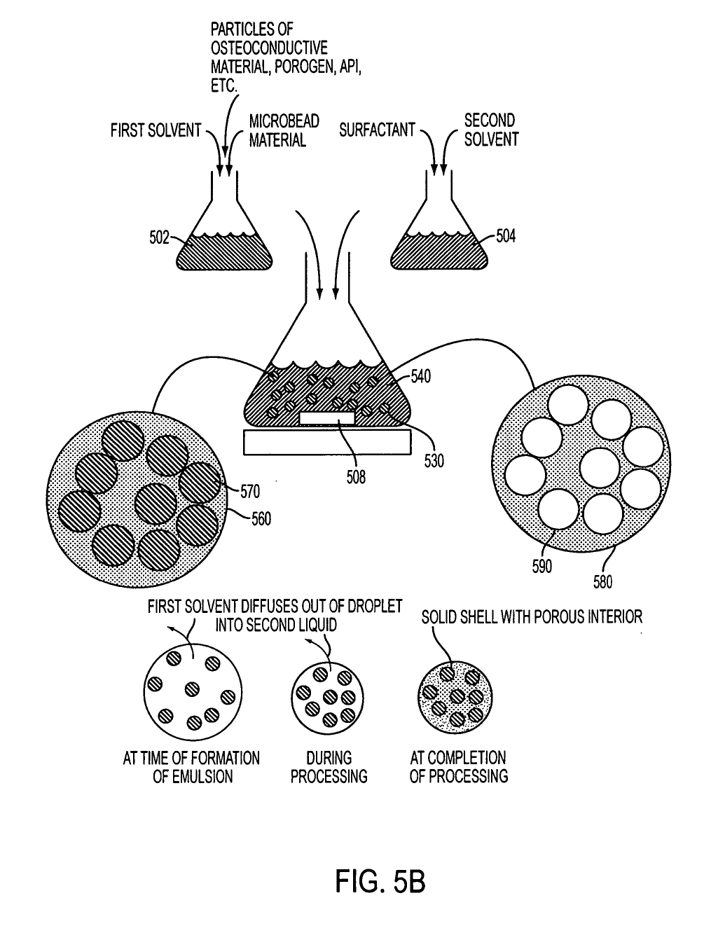

An aspect of the present invention is a method of three dimensional printing starting with the manufacture of the microbeads by an emulsion solvent extraction/evaporation process. A flowchart of this method is presented in FIG. 5a. Some of the steps are graphically illustrated in FIG. 5b.

The manufacture of the microbeads by the emulsion solvent extraction/evaporation process may start with a first solvent and a second solvent which are substantially immiscible with each other at approximately room temperature. The first solvent may be an organic solvent which may be substantially immiscible with water at approximately room temperature. The microbead material may be such that it dissolves in the first solvent but has little or no solubility in the second solvent. The microbead material may be or may include a polymer. The first liquid 502 may be formed by dissolving the microbead material such as a polymer in the first solvent. The first liquid does not have to be a saturated solution of the microbead material in the first solvent, although it could be. The first liquid may comprise an Active Pharmaceutical Ingredient or other bioactive substance dissolved in the first solvent, if it is desired that the eventual microbead contain an Active Pharmaceutical Ingredient or other bioactive substance.

The first solvent, which has been described as an organic solvent, could be a single substance which is an organic solvent, or it could include more than one organic solvent. If the first solvent is a single substance, it may for example be methylene chloride or chloroform, which are solvents for many polymers. The first solvent could be a mixture of these or any other suitable solvents. For example, petroleum ether is a mixture of various chemical species of similar structure but slightly different molecular weight. Another example of an organic solvent of interest is acetone, which is miscible with other organic solvents such as methylene chloride and chloroform, but also is miscible with water. Thus, for example, acetone can be included in the formulation along with a solvent such as methylene chloride to an extent such that the methylene chloride plus acetone will still be immiscible with the second liquid, and the acetone may help to dissolve solutes. However, with this combination of solvents, the acetone will relatively quickly pass out of the first liquid into the second liquid, leaving the solute behind inside the microbead. Such use of more than one organic solvent with differing properties can help to adjust the amount of solute contained inside the microbead, the rate of solidification of the microbead during the emulsion solvent extraction/evaporation process, etc.

The method of manufacturing the microbeads can further include mixing particles which are not very soluble in the first solvent into the first solution so that the first liquid is a suspension. Therefore, instead of the droplets being simply a solution of the polymer in the first solvent, the droplets may be a suspension of solid particles in a solution of the polymer in the first solvent. The solid particles may include particles of an osteoconductive substance (such as beta tricalcium phosphate, other members of the calcium phosphate family, other calcium compounds such as carbonates and sulfates, other ceramics, bioactive glass, etc.), and may include particles of API if the API is not sufficiently soluble in the first solvent, and may include particles of a porogen suitable to form pores within individual microbeads.

A second liquid 504 may comprise a second solvent, which may be water. The second solvent may further be selected so that the microbead material is not substantially soluble in the second solvent. Dissolved in the second solvent may be a surfactant. The surfactant may be selected so that it is primarily soluble in the second solvent, although it may have at least a slight extent of solubility in the first solvent. The surfactant may be a solid or a liquid. The surfactant may be chosen such that it is not highly soluble in the microbead material. The surfactant may be a member of the alcohol family. It may, for example, be polyvinyl alcohol (PVA).

The first liquid and the second liquid, after being individually prepared, may be combined with each other by any suitable means, which may include pouring one liquid into the other, etc., followed by agitating such as stirring, sonication, etc. so as to form an emulsion comprising droplets of the first liquid 530 dispersed in a continuous phase of the second liquid 540. The two liquids and their relative proportion may be selected such that when the two liquids are mixed together, the result is an emulsion having the form of droplets of the first liquid surrounded by a continuous phase of the second liquid. The size and size distribution of those droplets could be influenced by the viscosity of each of the two liquids and the relative proportion of the two liquids, all of which may be chosen to help produce droplets of the desired size or size range. The size and size distribution of the droplets of the first liquid in the continuous phase of the second liquid may be influenced by the vigorousness of agitation of the emulsion, and so the agitation characteristics also may be chosen appropriately to produce droplets of the desired size or size distribution. The mass of each individual droplet in which the first liquid exists in the second liquid at the time of creation of the droplet may typically be 10 to 50 times as large as the mass of the eventual microbead which is formed from the respective droplet.

If particles of osteoconductive material are present, it is believed that an individual droplet 560 and eventual microbead might contain anywhere from one to hundreds of particles 570 of osteoconductive material. If particles of porogen are used (for creating porosity within the eventual microbead), it is believed that a similar number of those particles may exist inside the droplet and eventual microbead. Of course, this would be influenced by the size of the particles of osteoconductive material which are supplied, or the size of the particles of porogen which are supplied, and the desired size of the microbead.

Still further, it is possible that the drops of the first liquid contained in the continuous phase of the second liquid may themselves be an emulsion, of drops of aqueous liquid 590 contained in a continuous phase of the organic solvent which in turn forms a droplet 580 in the overall first liquid 540. The aqueous liquid may contain an Active Pharmaceutical Ingredient or other bioactive substance which is at least somewhat water-soluble. In this embodiment, the overall situation during formation of the microbeads is a water in oil in water emulsion.

Another possibility is that the droplets of the first liquid in the second liquid may not be initially formed by mixing processes, which inherently have some degree of randomness and distribution of droplet sizes, but rather may be formed by dispensing discrete droplets of the first liquid which may have a fairly consistent droplet size, and introducing or injecting those droplets of the first liquid into the second liquid. Forming and introducing droplets may be accomplished, for example, by dispensing discrete droplets either continuously or on demand from a dispenser or printhead such as from a microvalve, from a piezoelectric dispenser, from a continuous jet either with or without piezoelectric stimulation, or by other means.

By this method droplets of the first liquid may be produced such that their size has a good degree of consistency and repeatability. In this situation, the processing parameters for the emulsion/evaporation process may be chosen such that the droplets as initially introduced into the second liquid would, to as great an extent as possible, persist through the rest of the microbead formation process, with a low probability of meeting up with and coalescing with other droplets, and with a low probability of breaking up into other droplets. For example, for this purpose it may be desirable to maintain the proportion of components in the overall emulsion such that the total volume of first liquid is relatively small compared to the total volume of the second liquid.

After the initial formation of the droplets by any method, agitation such as stirring or sonication could continue in order to maintain the existence of individual droplets, i.e., to discourage individual drops from coalescing to form larger drops. The presence, properties and concentration of the surfactant such as polyvinyl alcohol are also influential in this regard. The emulsion may be maintained, such as with agitation or stirring continuously applied, such as by magnetic stir bar 508, for a period of time sufficient for substantially all of the first solvent to exit from the droplets such as by diffusion. This time period may be several hours, or a day, or similar time period, depending on the size of the batch, diffusivities, concentrations and other factors. The first solvent may evaporate from the surface of the emulsion or second liquid as this process continues. The two solvents may be selected so that, at an operating temperature, the first solvent has a greater vapor pressure than the second solvent, which would encourage such preferential evaporation of the first solvent.

As a result of this process, the amount of the first solvent contained in individual droplets may gradually decrease with time until finally only solidified microbead material remains in the form of microbeads. Because of the fact that the solid microbead forms as a result of a gradual extraction of liquid from the droplets of the first liquid, and because droplets have a preferred essentially spherical shape, there is a tendency for the solid particles or microbeads to form with smoothly curved surfaces and to have approximately spherical shape.

By way of a non-limiting hypothesis, it is believed that at a certain stage, diffusion of solvent out of the microbead results in the creation of a hardened shell at the exterior the microbead, with some liquid remaining in the interior of the shell. By way of a further non-limiting hypothesis, it is believed that as further diffusion of solvent occurs out of the interior of the microbead, the microbead might approximately retain the exterior size and shape it had upon hardening of the shell, and might additionally acquire an interior which is hardened but somewhat porous. The rate at which the first solvent leaves the droplets may be influenced by physical properties and concentrations of the various substances, agitation parameters, temperature, vapor pressure of the vapor of the first solvent in the chamber where the process is taking place, and related physical parameters. By way of an additional hypothesis, it is believed that a relatively slow removal rate of the first solvent is more likely to result in smoothly-surfaced microbeads, and that a relatively fast removal rate of the first solvent is more likely to result in microbeads having a wrinkled surface.

After the microbeads have solidified, the microbeads may be collected by separation from the second liquid by collection means such as filtration and/or centrifuging; the microbeads may be washed with a secondary liquid phase such as with deionized water to remove any remaining surfactant from their surfaces; and the microbeads may be dried such as by lyophilization.

If solidified precursors of the completed microbeads comprise particles of a porogen, then at a subsequent timepoint the microbeads can be exposed to water or in general to a liquid which is a solvent for the porogen inside the microbeads. This can be done for a suitable time under suitable conditions (such as agitation and temperature) to substantially dissolve out the porogen from inside the microbeads, and then the microbeads can be dried. Alternatively, it is possible that porogen particles could be left inside the microbeads. In this case, if later steps associated with three dimensional printing result in any rearrangement of polymeric material, the porogen particles are still present to create pores, and may be leached out at a later timepoint.

Still other methods of manufacturing the microbeads are also included in the invention. Drops of the first liquid can be dispensed into air and may simply be allowed to solidify in air by evaporation of solvent. Appropriate ventilation systems and precautions may be provided to handle the vapor of the solvent. Again, drops may be formed by the use of a microvalve or a piezoelectric drop-on-demand dispenser to dispense discrete drops upon command, or drops may be formed by the breakup of a continuous jet, either with or without stimulation such as piezoelectric stimulation. For an Active Pharmaceutical Ingredient which has some solubility in water, air-drying may offer the advantage of eliminating the loss of API to the water phase which might occur during the emulsion solvent extraction/evaporation process. The evaporation rate of solvent from droplets which are drying in air may be controlled by controlling variables such as the vapor pressure of the solvent vapor in the vicinity of the evaporation, the temperature, etc.

Following manufacturing of the microbeads as described, the resulting dry powder may then be classified by size to remove large microbeads, allowing selection of microbeads having a desired size range. Classifying by size may be done by sieving, by air classifying, by liquid classifying, or by other sorting means. For applications involving three dimensional printing using microbeads as the powder, a desirable size of microbeads may be about 5 to about 150 micrometers in diameter. The actual range of sizes of powder particles may be more narrow than the numbers just given, but may be somewhere between the upper and lower values just given. The powder used for three dimensional printing may comprise the described microbeads, or the powder used for three dimensional printing may be a powder mixture which additionally comprises other microbeads, which may be different from the first microbeads, or may additionally contain other particles of any type. For bone-related applications, the other particles may include particles of an osteoconductive substance (such as tricalcium phosphate, other calcium phosphates, other calcium compounds, other ceramics, bioactive glass, etc.), particles of a porogen, particles of a bioactive substance, or a combination thereof. Other than porogens (which will not be present in a finished biostructure) and optionally API, the particles may be sufficiently large so as to avoid causing a macrophage response.

Three dimensional printing includes depositing a layer of powder or powder mixture. Powder deposition may be accomplished by roller spreading, by slurry deposition, or by other suitable means. Then, in selected places, a binder liquid may be deposited onto the powder suitably to bind powder particles to each other and to already-bound powder particles. Then another layer of powder may be deposited and the process may be repeated for as many layers as needed to manufacture a desired three-dimensional shape. Unbound powder supports bound powder during printing and is later removed. After removal of unbound powder, it is possible to perform post-processing steps which may include infusing desired substances such as biologically useful substances into pores in the article made by three dimensional printing.

There are two principal ways by which powder particles may be bound together in three dimensional printing. One is by dissolution of powder followed by resolidification, if the binder liquid is a solvent for at least some of the powder particles. For example, the dispensed binder liquid may comprise chloroform, which is capable of dissolving many polymers, or methylene chloride, or non-halogenated solvents such as tetrahydrofuran, ethyl acetate, acetonitrile and acetone. If the binder liquid used in 3DP is a solvent for the powder particles, it does not have to be the same solvent which was used as the first solvent in the emulsion solvent extraction/evaporation process. The binder liquid in this case could have still other substances dissolved in it, such as Active Pharmaceutical Ingredients. Alternatively, the binder liquid may be a non-solvent for at least some of the powder particles but may have a binder substance dissolved in the binder liquid, such that when the binder substance remains behind after evaporation of the volatile part of the binder liquid, the powder particles are bound together by the binder substance. In this event, typically the binder liquid may be an aqueous solution. If the powder layer contains powder particles in addition to the described microbeads, it is possible that either or both, or neither, type of powder particle and/or microbead (in any combination) may be soluble in the binder liquid or in a particular binder liquid. Different binder liquids could be used in different places within the 3DP manufacturing process, to impart different local composition or characteristics to the article manufactured by 3DP. More than one type of powder particle or microbead can be present in the powder mixture or the powder bed in different places. As described herein, one type of microbead may be chosen so that it is more likely to soften and flow or dissolve in a particular solvent upon exposure to certain processing conditions while another type of microbead may be chosen so that it is more likely to remain intact under those same processing conditions. The latter type of microbead may, for example, contain a bioactive substance which is at least somewhat water-soluble. The composition of the powder mixture can differ from place to place within the biostructure. It is also possible that three dimensional printing could be performed as described in a copending patent application entitled Manufacturing Process, such as Three Dimensional Printing, Including Binding of Water-Soluble Material Followed by Softening and Flowing and Forming Films of Organic-Solvent-Soluble Material, filed May 12, 2005, which is herein incorporated by reference in its entirety. The three dimensional printing may be performed so as to result in macrochannels or other complex geometric features as described elsewhere herein.

A particular category of polymers which are useful in biomedical applications is comb polymers, which have specific properties useful for either encouraging or preventing the adhesion of specific types of cells as described in U.S. Pat. No. 6,150,459. Like other polymers, comb polymers can dissolve in certain solvents such as organic solvents. At least some comb polymers have a property of migrating to or collecting preferentially at a free surface of an article during solidification. The method of the present invention can include dissolving comb polymers in the first solvent in the emulsion solvent extraction/evaporation process described elsewhere herein, either alone or in combination with other polymers or other substances. Articles of the present invention can be microbeads which are made entirely of comb polymers. Articles of the present invention can be microbeads which contain both comb polymers and non-comb (ordinary) polymers in such a distribution that there may be a relatively larger concentration of comb polymer at the surfaces of the microbeads and a relatively smaller concentration of comb polymer in the interior of the microbeads. The microbeads used as the powder in three dimensional printing do not all require identical composition. For example, some of the microbeads could contain comb polymer while others do not.

In the case of microbeads containing comb polymer, an article may comprise a plurality of microbeads joined to each other, with at least some of the microbeads having a concentration of comb polymer which is greater at the surface of the microbead than in the interior of the microbead. Microbeads may be joined to each other by neck regions, and the neck regions may have a concentration of comb polymer which is greater at the surface of the neck than in the interior of the neck.

A biostructure may be made, using three-dimensional printing techniques, starting with any of the microbeads described herein as the powder or as a component of the powder mixture which is spread or deposited to form the successive layers of powder in the 3DP process. The possible biostructures which may be so made include a bone replacement, a tissue scaffold, a drug delivery device, and other devices for biological applications.

The invention is further described, but is in no way limited, by the following non-limiting examples.

### EXAMPLES

### Example 1

A batch of polymer microbeads was prepared as follows. The first solvent was methylene chloride, and the second solvent was water. A first liquid was prepared by dissolving 6.944 grams of polycaprolactone (PCL) of Molecular Weight 85,000 Daltons (Sigma-Aldrich, St. Louis, Mo.) in 198.81 grams of methylene chloride (Sigma-Aldrich). This gave a concentration of PCL of 3.375% by weight. A second liquid was prepared by dissolving 5.101 grams of polyvinyl alcohol (PVA) 87-89% hydrolyzed, Molecular Weight 13,000 to 23,000 Daltons (Sigma-Aldrich) in 1020.2 grams of deionized water. The two liquids were then combined and agitated to form an emulsion of drops of the first liquid surrounded by a continuous phase of the second liquid. The emulsion was stirred using a magnetic stir bar at 200 rpm for 8 hours. This time period allowed the methylene chloride to diffuse out of the droplets into the polyvinyl alcohol dissolved in the water and then to evaporate from the open surface of the water-PVA solution. At the conclusion of the stirring, microbeads of solid polycaprolactone remained, suspended in the remaining liquid. These microbeads were removed from the remaining liquid by filtering and then were washed three times each with 300 cc of deionized water to remove any remaining PVA. The filter cake was then frozen at −70° C. and was lyophilized to remove all remaining water. The result was a free-flowing polymer microbead powder.

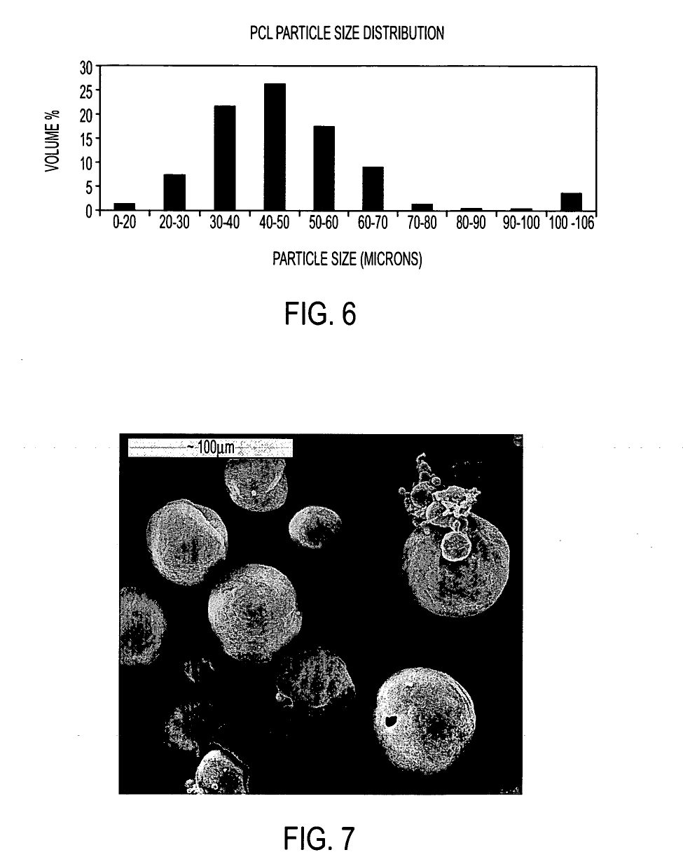

This powder was sieved with a 106 micron sieve to remove any large particles, which would be considered undesirable for a particular three dimensional printing application. The mass of powder which passed through the sieve and therefore was considered useful amounted to 5.46 g, or 79% of the starting PCL material. The sieved powder was tested in a Horiba particle size analyzer (Horiba Instruments, Ann Arbor, Mich., Model CAPA-700) in gravitational settling mode using water as the dispersant, to characterize its particle size distribution. The median particle size was 49 microns. FIG. 6 is a graph of the particle size distribution of the polymer powder. FIG. 7 is a Scanning Electron Microscope micrograph of some of the powder particles.

### Example 2

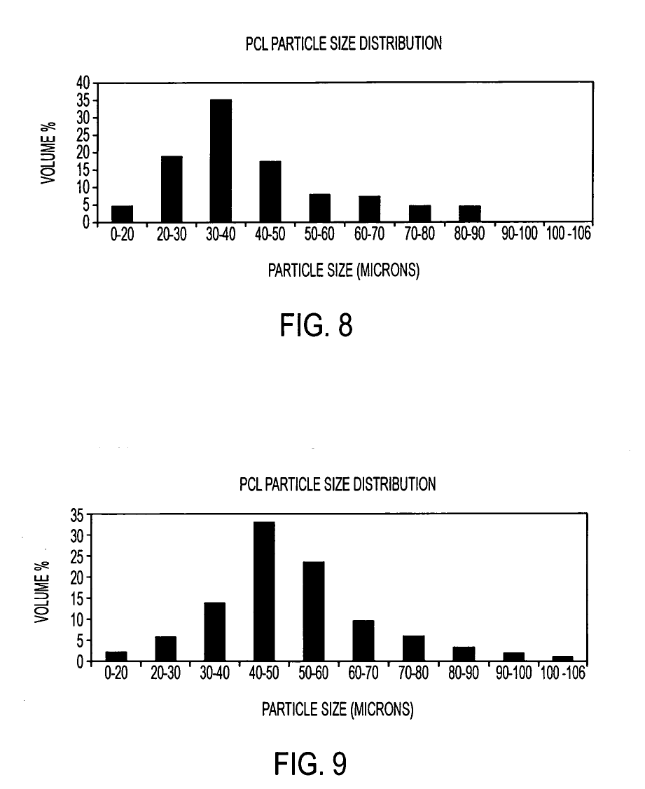

This example repeats the process of Example 1, except that the batch size was larger and accordingly a longer time was used. In this example the batch of the first liquid was prepared using 24.89 grams of PCL dissolved in 712.57 grams of methylene chloride. The second liquid was prepared by dissolving 15.097 grams of PVA in 2986.9 grams of deionized water. The two liquids were mixed together and emulsified and the emulsion was stirred for 24 hours at 400 rpm using a Labmaster stirrer (SPX Corp., Wytheville, Va., Model No. 223116 fitted with a fan-type impeller, SPC Corp. Part No. A310). Then, similar to the previous Example, the microbeads were filtered and washed four times with 500 cc of deionized water. The filter cake was frozen at −70° C. and then lyophilized until dry. The powder was then sieved with a 106 micron screen, and the powder which passed through the 106 micron screen amounted to 22.48 g, resulting in a yield of 90% of the amount of PCL originally processed. The particle size distribution is shown in FIG. 8.

### Example 3

Example 3 illustrates the process of the present invention applied to a polymer of a somewhat smaller molecular weight than in Examples 1 and 2. In this example, the Molecular Weight of the PCL polymer was about 65,000 Daltons (Sigma-Aldrich, Milwaukee, Wis.). In general, the viscosity of the first liquid is known to influence the size of the droplets formed in the emulsion. In using this different molecular weight PCL, it was found that a different concentration of this polymer in methylene chloride could be used to result in approximately the same viscosity of the first liquid (the polymer solution) as for the solution which was used in Examples 1 and 2. For the selected polymer, PCL having a Molecular Weight of 65,000 Daltons, it was found that the viscosity of a 5 w/w % solution of this polymer was similar to the viscosity of the 3.375 w/w % PCL 85,000 Molecular Weight solution which was used in Examples 1 and 2. The agitation and other processing steps were performed similarly to the steps performed in Examples 1 and 2. The following quantities and components were used: 41.873 grams of PCL having a Molecular Weight of 65,000 Daltons, 795.66 grams of methylene chloride, 16.477 grams of PVA, and 3295.5 grams of deionized water. Stirring, filtration, washing, lyophilization, and sieving were the same as in Example 2. The recovered yield of particles smaller than 106 microns was 38.50 grams, or 92% of the original mass of PCL. FIG. 9 shows the particle size distribution of the particles.

### Example 4

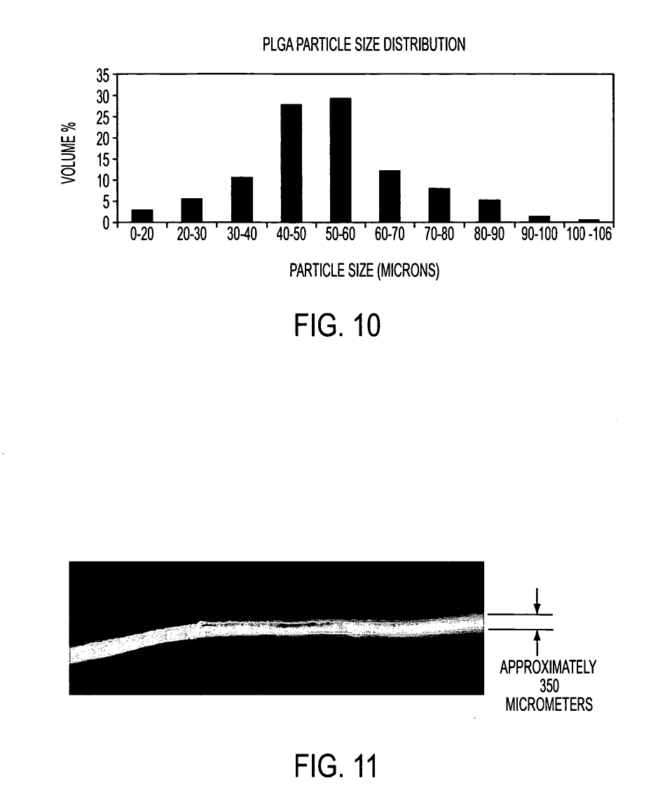

In Example 4, a different polymer was used, namely polylactic co-glycolic acid (PLGA). The PLGA used had a Molecular Weight of approximately 60,000 Daltons (RG755) and was obtained from Boehringer Ingleheim (Ridgefield, Conn.). The first liquid was formed by dissolving 13.7 grams of PLGA polymer in 259.61 grams of methylene chloride, which produced a solution of approximately the same viscosity as the solutions in Examples 1-3. The second liquid was made by dissolving 5.398 grams of PVA in 1074.2 grams of deionized water. The stirring, filtration, washing, lyophilization, and sieving were the same as in Example 1. The yield of particles smaller than 106 microns was 12.589 grams, giving a fractional yield of 92%. The particle size distribution resulting from this Example is shown in FIG. 10.

All of the particle size distributions in these first four Examples are fairly similar to each other.

### Example 5

The present invention has also demonstrated the ability to incorporate biologically active comb polymers. It is believed that these polymers, because of their unique structure, preferentially migrate to the surface of the emulsion droplets during the process of the present invention. It is believed that this leaves the comb polymer embedded in the surface of the microbeads with the polyethylene oxide side chains presented on the surface of the microbead. This is believed to allow tailoring of the surface properties of the powder particles, which may be a desirable feature for articles manufactured by three dimensional printing for biological use.

In this Example, the method of the present invention was performed using two polymers both dissolved in the first solvent to form the first liquid. One of the polymers was PCL with a molecular weight of 85,000 Daltons. 8.55 grams of it were used. The other polymer was 0.176 grams of comb polymer labeled with anthracene as a marker substance. The comb polymer was soluble in methylene chloride and readily mixed with methylene chloride that already had PCL dissolved in it. Both of these polymers were dissolved in 244.94 grams of methylene chloride. The second liquid comprised 5.095 grams of PVA dissolved in 1013.8 grams of deionized water. Stirring, filtration, washing, lyophilization, and sieving were the same as in Example 1. It is believed that the comb polymer was concentrated at the surface of the microbeads.

### Example 6

It would also be possible to reverse the roles of the organic solvent and the water and to produce microbeads of a primarily water-soluble substance rather than microbeads of a substance which is primarily soluble in an organic solvent. In such a process, the first solvent would be water and the microbead material could be a water-soluble substance such as any water-soluble salt or a sugar. The second solvent could be an oil such as vegetable oil or in general any light oil, possibly chosen so that it is less easily evaporable than water (has a lower vapor pressure than water at the operating temperature). The volume of the oil would be at least several times as large as the volume of the water. The miscibility agent could be a substance which is primarily miscible with oil but has some miscibility with water, such as butyl alcohol or another short-chain-length aliphatic alcohol.

### Example 7

Three dimensional printing was performed onto a powder bed comprising microbeads of polycaprolactone which had been made by the described emulsion solvent extraction/evaporation process. Chloroform was dispensed through a printhead onto powder beds. The printhead used a microvalve (The Lee Company, Westbrook, Conn.) at a dispense rate of 800 drops/second. A simple shape was formed by dispensing droplets of chloroform in a single line onto the powder bed. In this Example, this was done only on a single layer of powder for only a single isolated line, thereby producing the simplest possible shape. The shape thus produced is shown in FIG. 11.

### Example 8

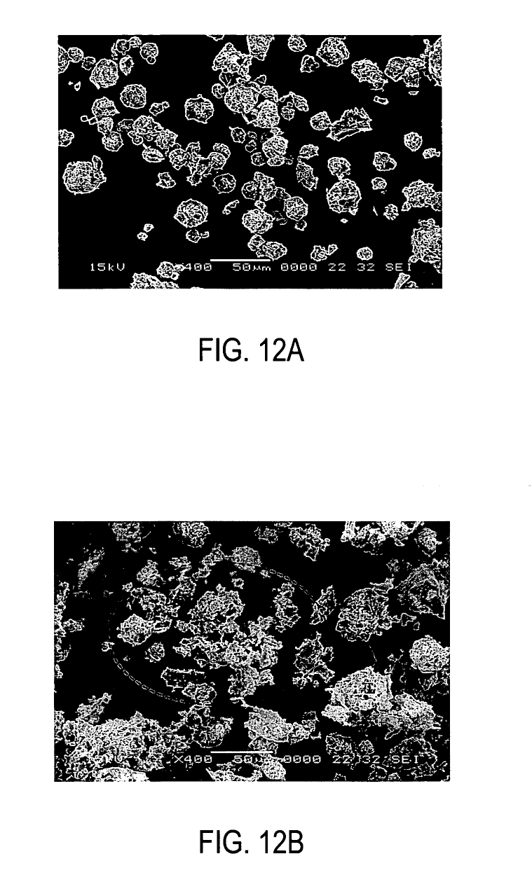

An experiment was performed which started with particles of a commercially available beta-TCP powder, subjected those particles to a series of steps described herein, and finally isolated and examined those TCP particles. The TCP particles were particles of a commercially available beta-TCP powder (from Tomita Pharmaceutical Co., Ltd,, Tokyo, Japan). These particles had been manufactured by spray-drying and therefore it is believed that the particles were porous or hollow. These particles had been pre-sintered at a moderate temperature but were not especially robust. These particles had an average particle size of either approximately 20 micrometers or approximately 40 micrometers (depending on a particular batch or experiment) as ascertained by liquid classifying. These particles are shown in FIG. 12a.

The polymer used was either PCL or PLGA. A solution of polymer in methylene chloride was created, and the just-described particles of TCP were mixed in with the solution and were stirred to form a suspension. Then, that suspension was mixed with water to form an emulsion of drops of the suspension surrounded by a continuous phase of the water. That suspension was continuously stirred until the methylene chloride diffused out and microbeads solidified. The microbeads were separated and dried and used to form a powder mixture which also contained particles of TCP and particles of a porogen. This powder mixture was used in three dimensional printing to make simple cubical porous shapes by dispensing water as a binder liquid followed by softening and flowing of the polymer (due to exposure to chloroform vapor) to form polymeric films which also held the TCP particles. This process is described in a co-pending patent application entitled Manufacturing Process, such as Three Dimensional Printing, Including Binding of Water-Soluble Material Followed by Softening and Flowing and Forming Films of Organic-Solvent-Soluble Material, filed May 12, 2005, which is herein incorporated by reference in its entirety. This was followed by leaching of the sugar porogen particles. Later, to ascertain the state of the TCP particles after all of the processing steps up until that point, the printed articles were immersed in methylene chloride so that the polymer dissolved out and the TCP particles were separated and collected (by filtration). The result is shown in FIG. 12b. It is believed that the original TCP particles were not particularly robust. It was found that after the completion of all these process including the separation, there were particles of TCP which were smaller than any of the TCP particles which had been originally supplied to form the powder mixture. Some of these particles or fragments were small enough so that they would have induced a macrophage response in the body of a recipient, which is of course undesirable. It is believed that some fracturing of particles occurred during the various manufacturing steps, which produced the undesirably small particles. Separate experiments were conducted which simulated some individual steps of the just-described sequence of manufacturing steps to try to ascertain when the fracturing might have occurred. It appears that merely exposing these particular TCP particles to polymeric solution can cause fracturing, and that the fracturing can occur even without the agitation experienced during emulsion processing, and even without the powder spreading and other processes that occur during three dimensional printing. It is believed that the fracturing occurred simply due to shear stress resulting from exposure to or creation of the polymeric solution.

### Example 9

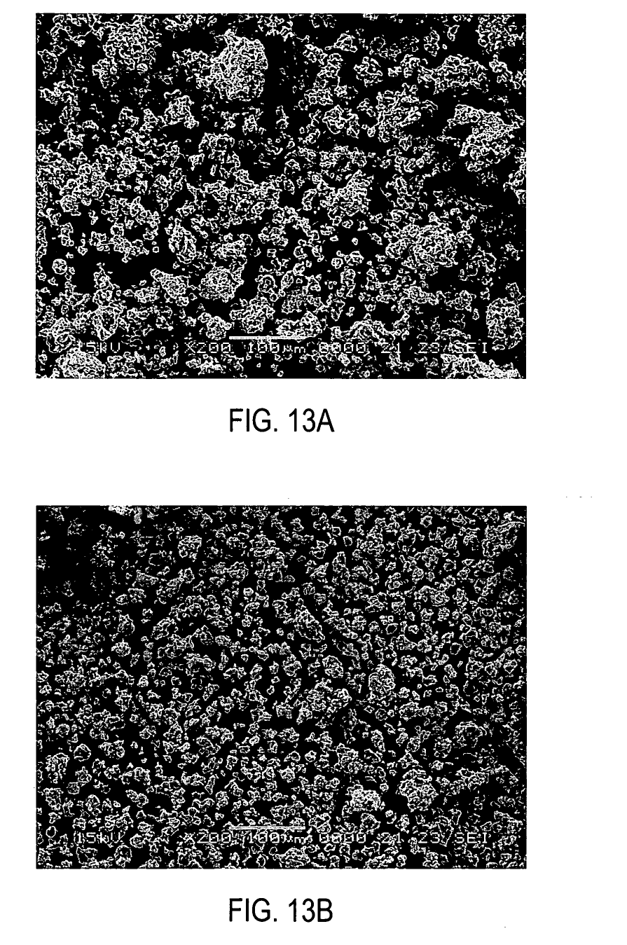

Accordingly, another experiment was performed involving beta-TCP particles which were believed to be more robust than the particles used in Example 8. These particles were beta-TCP which were already part of a finished product and hence had undergone a substantial amount of sintering (such as 1200 C for 2 hours). The product was crushed and ground (comminuted) to provide the particles used in this experiment. The particles were then size-classified by sieving so as to insure that all particles used were in a desired size range. Such particles are pictured in FIG. 13a. As in the previous Example, the particles were used in the emulsion process to make microbeads which contained the TCP powder, and particles were further mixed with polymeric microbeads and porogen particles to form the powder mixture upon which three dimensional printing was performed. This was followed by softening and flowing of the polymer. After completion of all of the processes as described in the preceding Example, analysis of the TCP particles revealed that these particles were not nearly so damaged by the various processes as were the particles of Example 8. FIG. 13b shows particles of this type after processing to make a simple porous cube, further followed by dissolving out the polymer and isolating the TCP particles, just as was done in the preceding Example. It is believed that the particles in this Example survived better than the particles in the preceding Example. It is believed that the particles in this Example are more robust because they had been more thoroughly sintered (e.g., approximately 1200 C for 2 hours) than the particles of Example 8. It is also believed that these particles were more dense than the particles of Example 8.

### Example 10

Yet another possibility for providing TCP particles which are part of microbeads and/or part of the powder mixture for three dimensional printing is that the TCP particles are granules prepared by a granulating process followed by sintering.

In a non-limiting exemplary granulation technique, powder(s) of interest is processed in a fluidized bed granulator in which the powder is fluidized by upwardly moving air, while a binder liquid, such as an aqueous solution of polyacrylic acid, is sprayed into the fluidized powder, thereby causing agglomeration of individual powder particles of interest to each other or to other agglomerates. The longer the process is carried out, the larger the agglomerates become, on average. The process is carried out for a suitable length of time to produce desirably sized agglomerates.

The powder particles which are supplied to the granulator may comprise precursors for a desired final ceramic substance. For example, the powder may contain hydroxyapatite and calcium pyrophosphate (which may be obtained from Cosmocel, Monterrey, Nuevo León, México) so as to produce tricalcium phosphate (TCP) upon reaction at elevated temperature. Alternatively, or in addition thereto, the powder particles supplied to the granulator may contain particles of a desired final substance already in its final chemical form.

In addition, the powder supplied to the granulator may further include one or more porogens that may create desired porosity in the eventual granules. Non-limiting examples of porogens useful with this invention include lactose and, in general, almost any solid, decomposable, particulate organic substance. Lactose decomposes into gaseous decomposition products when heated to about 220° C., resulting in voids where the lactose formerly existed. In one embodiment of the invention, the combined powder used for the fluidized bed granulation process may comprise about 40% by weight lactose and about 60% by weight precursors of tricalcium phosphate.

Following granulation, the agglomerates are subjected to sintering to form finished granules. Prior to reaching sintering temperatures, the porogen (such as, for example, lactose) and the binder substance (such as, for example, polyacrylic acid) decompose into gaseous decomposition products resulting in void spaces. At temperatures above those necessary to decompose the porogen and binder substances, a reaction of the precursors occurs to form a desired final ceramic product, for example TCP, if the agglomerates contain combinations of precursor substances suitable to react with each other.

At appropriate temperatures, individual powder particles within the granules may sinter to each other. Sintering processes are performed at peak temperatures of about 1200° C. for about 2 hours. Sintering may result in granules which contain a number of the original powder particles joined to each other, while also containing pores some of which may result from decomposition of the porogen.

The resulting granules are used as particles which can be included within microbeads or which can be mixed with microbeads of the present invention to form a powder mixture which can be printed upon during three dimensional printing. It is expected that the robustness of particles prepared by this method would more resemble the robustness of the hand-crushed particles of Example 9, rather than the robustness of the spray-dried particles of Example 8.

### Example 11

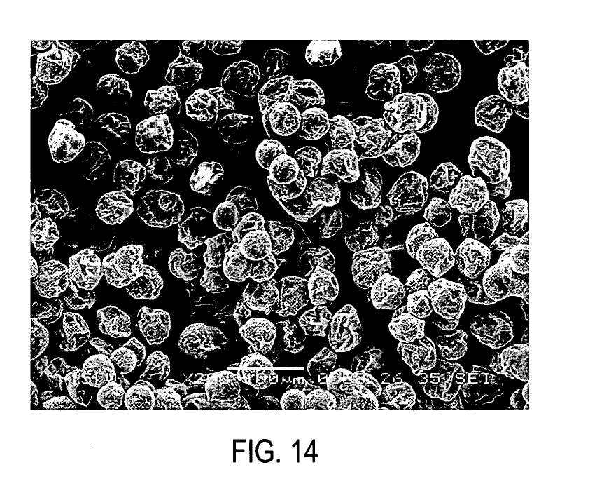

Microbeads were made by a continuous-jet printhead dispensing into air, with the solvent evaporating into air. A photomicrograph of such microbeads is shown in FIG. 14. These microbeads are not joined to each other; they are merely an aggregate. They are made of PCL polymer which was dissolved in methylene chloride solvent at a concentration of 1% by weight. Upon evaporation of the solvent, the droplets became the size illustrated, and also acquired somewhat wrinkled surfaces. It is believed that the wrinkled surfaces may be due partly to the relatively fast rate at which evaporation of the solvent occurred. The evaporation of the solvent in air took minutes, as compared to hours for the microbead solidification in the emulsion process. The wrinkled surfaces may also be due partly to the fact that the solution was fairly dilute and a substantial amount of solvent had to evaporate relative to the amount of mass eventually left in the microbead. It is believed that use of a more concentrated solution initially might result in a less wrinkled surface.

Summary and Advantages and Further Comments

In three dimensional printing, the use of substantially spherical particles of closely controlled size may be advantageous for powder layer deposition and other steps. The method of the present invention offers several advantages over conventional milling, for the creation of small particles or microbeads such as microbeads of polymer. The batch size of the emulsion solvent extraction/evaporation process is limited only by the size of the vessel in which the emulsion is stirred. Thus, large batches are possible, such as approximately 1 kg per batch, which can provide much larger production rates than a milling process. Furthermore, of the particles which are obtained from the emulsion solvent extraction/evaporation process, typically a much larger fraction of them are usable, in the sense of having good shape and desired size, than would be obtained from milling processes. With the method of the present invention, typically 75% to 90% of the particles are suitably sized for three dimensional printing, as compared to less than 20% yield when particles were produced by a milling process. This is a significant improvement over the yield of desirably sized particles obtained from the milling process. Furthermore, the shapes of particles from this process are closer and more consistently closer to spherical than the shapes of particles obtained by milling.

In three dimensional printing, the use of nearly spherical particles of closely controlled size is believed to result in better spreading of powder and better control over the size of pores in three dimensionally printed articles.

In addition to using such microbeads as the starting powder in three dimensional printing, it is possible to use microbeads made in accordance with the present invention in applications other than three dimensional printing, such as forming articles by pressing or otherwise joining or partially joining powder particles together. In addition, it is possible to use microbeads made in accordance with the present invention in the method using a powder mixture with water printing followed by softening and flowing, as described in patent application. Any of the microbeads of the present invention can be so used. More than one of the above kinds of microbeads could be combined in a powder mixture, in any combination. In addition to the microbead manufacturing methods described herein, microbeads could also be prepared by polymerization, by single/double emulsion solvent evaporation/extraction, by phase separation.

The microbeads of the present invention can provide both osteoconductivity and also the ability to stimulate the formation of bone, which is a desirable combination. Other bioactive substances can also be delivered via the same microbeads. Any of these can be released in a controlled manner depending on the degradation of the resorbable polymer. Aggregates (powder mixtures) can offer the advantages of the described microbeads together with the properties of other substances whose particles may be mixed into the powder mixture, thereby providing significant control over properties of the resulting biostructure. Of course, detailed geometric fabrication ability is also available from the three dimensional printing process.

The above description of various illustrated embodiments of the invention is not intended to be exhaustive or to limit the invention to the precise form disclosed. While specific embodiments of, and examples for, the invention are described herein for illustrative purposes, various equivalent modifications are possible within the scope of the invention, as those skilled in the relevant art will recognize. The teachings provided herein of the invention can be applied to other purposes, other than the examples described above.

The various embodiments described above can be combined to provide further embodiments. Aspects of the invention can be modified, if necessary, to employ the process, apparatuses and concepts of the various patents, applications and publications described above to provide yet further embodiments of the invention. All patents, patent applications and publications cited herein are incorporated by reference in their entirety. Certain methods for performing three dimensional printing, and articles and attributes produced thereby, are disclosed in U.S. patent application Ser. No. 10/122,129, filed Apr. 12, 2002, the disclosure of which is herein incorporated by reference in its entirety.

These and other changes can be made to the invention in light of the above detailed description. In general, in the following claims, the terms used should not be construed to limit the invention to the specific embodiments disclosed in the specification and the claims, but should be construed to include all methods of three dimensional printing, methods of microbead manufacture, articles of manufacture and apparatus that operate under the claims. Accordingly, the invention is not limited by the disclosure, but instead the scope of the invention is to be determined entirely by the following claims.

Any technique described for any individual stage of the processing could be used with any other technique for any other stage, in any combination.

The invention may be practiced in ways other than those particularly described in the foregoing description and examples. Numerous modifications and variations of the invention are possible in light of the above teachings and, therefore, are within the scope of the appended claims.

The entire disclosure of each document cited (including patents, patent applications, journal articles, abstracts, manuals, books, or other disclosures) in the Background of the Invention, Detailed Description, and Examples is herein incorporated by reference in their entireties.

While the invention has been described with reference to particularly preferred examples and embodiments, those skilled in the art will appreciate that various modifications may be made to the invention without departing from the spirit and scope thereof.

What is claimed is:

1. A method of manufacturing a biostructure, the method comprising forming a first liquid by dissolving a microbead material in a first solvent, creating an emulsion comprising droplets of the first liquid surrounded by a continuous phase of a second liquid which is substantially immiscible with the first liquid, maintaining the emulsion for a sufficient period of time for substantially all of the first solvent to exit from the droplets of the first liquid, resulting in the droplets becoming solidified microbeads, collecting the microbeads, depositing a layer of powder comprising the microbeads, depositing onto the layer of powder in selected locations a binder liquid suitable to cause at least some of the powder in the selected locations to join together, and repeating the layer-depositing and binder-liquid-depositing steps as many times as needed to manufacture the biostructure.

2. The method of claim 1, wherein the first solvent is an organic solvent which is substantially immiscible with water at room temperature, and wherein the second liquid comprises water.

3. The method of claim 2, wherein the organic solvent comprises a solvent selected from the group consisting of methylene chloride, chloroform, and petroleum ether.

4. The method of claim 2, wherein the second liquid further comprises a surfactant.

5. The method of claim 1, wherein the microbead material comprises at least one polymer which is resorbable in the human body.

6. The method of claim 1, wherein the microbead material comprises at least one polymer selected from the group consisting of: Polylactides; Polyglycolides; Epsilon-caprolactone; Polyhydroxyvaleric acid; Polyhydroxybutyric acid; other Polyhydroxy acids; Polytrimethylene carbonate, polyamines, vinyl polymers, Polyacrylic acid and their derivatives including esters; Polyethylene glycols; Polydioxanones; Polycarbonates; Polyacetals; Polyorthoesters; Polyamino acids; Polyphosphoesters; Polyesteramides; Polyfumerates; Polyanhydrides; Polycyanoacrylates; Poloxamers; Polyurethanes; Polyphosphazenes; Aliphatic polyesters; Poly(amino acids); Copoly(ether-esters); Polyalkylene oxalates; Polyamides; Poly(iminocarbonates); Polyoxaesters; Polyamidoesters; Polyoxaesters containing amine groups; Polyacetals; Polyalkanoates; Gelatin; Collagen; Elastin; Polysaccharides; Alginate; Chitin; Hyaluronic acid; and copolymers and terpolymers of any combination of any of these substances.

7. The method of claim 1, wherein forming the first liquid further comprises dissolving or mixing into the first solvent a bioactive substance.

8. The method of claim 7, wherein the bioactive substance stimulates the formation of bone.

9. The method of claim 7, wherein the bioactive substance is lovastatin, or another member of the statin family, or another HMG-CoA reductase inhibitor, or a growth factor.

10. The method of claim 7, wherein the bioactive substance comprises an angiogenic substance, an antibiotic or an anesthetic or a chemotherapeutic agent.

11. The method of claim 1, wherein forming the first liquid further comprises mixing into the first solvent particles of an osteoconductive material.

12. The method of claim 1, wherein forming the first liquid further comprises mixing into the first solvent particles of a porogen.

13. The method of claim 12, further comprising, after collecting the microbeads, dissolving the porogen out of the microbeads.

14. The method of claim 1, wherein the creating the emulsion comprises introducing the first liquid and the second liquid to each other and agitating the liquids sufficiently to create the emulsion.

15. The method of claim 1, wherein the creating the emulsion comprises creating discrete drops of the first liquid and introducing the discrete drops into the second liquid.

16. The method of claim 1, further comprising, after the collecting but before the depositing of the layer of powder, selecting microbeads having a size which is within a desired size range.

17. The method of claim 1, further comprising, after the collecting of the microbeads but before the depositing the layer of powder, mixing the microbeads with other particles to form the powder.

18. The method of claim 17, wherein the other particles comprise particles of osteoconductive material.

19. The method of claim 18, wherein the particles of osteoconductive material are formed by processes which include sintering.

20. The method of claim 17, wherein the other particles comprise particles of a water-soluble porogen.

21. The method of claim 1, wherein the repeated depositing of the layers of powder comprises depositing powder having different characteristics in different places.

22. The method of claim 1, wherein the depositing the binder liquid comprises depositing a binder liquid which is capable of dissolving at least some of the powder.

23. The method of claim 1, wherein the depositing the binder liquid comprises depositing a binder liquid which is capable of dissolving at least some of the powder and which also contains some additional substance dissolved in it.

24. The method of claim 1, further comprising, after all the other steps, infusing the article with one or more biologically useful substances.

25. A biostructure made by the method of claim 1.

26. A biostructure comprising particles of at least one osteoconductive substance, the particles having a particle size distribution in which the particle size distribution is bimodal and in which substantially all of the particles are larger than a minimum particle size to avoid a macrophage response, the biostructure further comprising a polymer-containing network which holds the particles and is porous.

27. The biostructure of claim 26, wherein the osteoconductive substance comprises one or more substances selected from the group consisting of beta tricalcium phosphate, other members of the calcium phosphate family, calcium sulfates, calcium carbonates, other calcium compounds, other ceramics, bioactive glass, and combinations thereof.

28. The biostructure of claim 26, wherein the biostructure further comprises a bioactive substance which stimulates the formation of bone.

29. The biostructure of claim 28, wherein the bioactive substance is lovastatin, or another member of the statin family, or another HMG-CoA reductase inhibitor, or a growth factor.

30. The biostructure of claim 26, wherein the polymer is resorbable in the bodily environment at a rate which produces a desired release characteristic of the bioactive substance.

31. The biostructure of claim 26, wherein the biostructure further comprises macrochannels.

32. A biostructure comprising a plurality of microbeads each joined to other microbeads or to first particles of an osteoconductive substance, wherein at least some of the microbeads comprise: a polymer, and a bioactive substance which stimulates the formation of bone, and second particles of an osteoconductive substance.

33. The biostructure of claim 32, wherein the bioactive substance is lovastatin, or another member of the statin family, or another HMG-CoA reductase inhibitor, or a growth factor.

34. The biostructure of claim 32, wherein the osteoconductive substance comprises one or more substances selected from the group consisting of beta tricalcium phosphate, other members of the calcium phosphate family, calcium sulfates, calcium carbonates, other calcium compounds, other ceramics, bioactive glass, and combinations thereof.

35. The biostructure of claim 32, wherein the polymer is resorbable in the bodily environment at a rate which produces a desired release characteristic of the bioactive substance.

36. The biostructure of claim 32, wherein the biostructure further comprises macrochannels.

37. A microbead comprising: a polymer, and one or more particles of a first osteoconductive substance, and a bioactive substance which stimulates the formation of bone.

38. The microbead of claim 37, wherein the bioactive substance is lovastatin, or another member of the statin family, or another HMG-CoA reductase inhibitor, or a growth factor.

39. The microbead of claim 37, wherein the first osteoconductive substance comprises one or more substances selected from the group consisting of beta tricalcium phosphate, other members of the calcium phosphate family, calcium sulfates, calcium carbonates, other calcium compounds, other ceramics, bioactive glass, and combinations thereof.

40. The microbead of claim 37, wherein the polymer is resorbable in the bodily environment at a rate which produces a desired release characteristic of the bioactive substance.

41. The microbead of claim 37, wherein the microbead has internal porosity.

42. The microbead of claim 37, further mixed together with additional such microbeads and with additional particles which are not microbeads, to form an aggregate.

43. The aggregate of claim 42, wherein substantially all of the microbeads and particles of the aggregate are suitably sized for spreading during three dimensional printing.

44. The aggregate of claim 42, wherein substantially all of the microbeads and particles of the aggregate are between a maximum size and a minimum size, the ratio of the maximum size to the minimum size being less than approximately 5.

45. The aggregate of claim 42, wherein the additional particles comprise a porogen.

46. The aggregate of claim 42, wherein the additional particles comprise a second osteoconductive substance.

47. The method of claim 1, wherein forming the first liquid comprises creating an emulsion of droplets of aqueous liquid surrounded by a continuous phase which comprises the first solvent.

48. The biostructure of claim 32, further comprising microbeads which contain aqueous droplets which contain at least one water-soluble bioactive substance.

49. The aggregate of claim 42, further comprising microbeads which contain at least one water-soluble bioactive substance.

50. The aggregate of claim 42, further comprising microbeads which contain aqueous droplets which contain at least one water-soluble bioactive substance.
